(19) United States
(12) Patent Application Publication
(10) Pub. No.: US 2005/0106274 A1
(43) Pub. Date: May 19, 2005

(54) Compositions for oral health in animals

(76) Inventor: Deborah Overoyen, Doha (QA)

Correspondence Address: FISH & RICHARDSON P.C., 3300 DAIN RAUSCHER PLAZA, 60 SOUTH SIXTH STREET, MINNEAPOLIS, MN, 55402, US

(21) Appl. No.: 10/715,583

(22) Filed: Nov. 18, 2003

(51) Int. Cl. A61K 35/78

(52) U.S. Cl. 424/769

(57) ABSTRACT

The invention provides for S. persica compositions and articles of manufacture, as well as products containing an extract of S. persica. The compositions, articles or manufacture, and products are used for the oral health of companion animals.

### TECHNICAL FIELD

This invention relates to oral health, and more particularly to compositions that improve the oral health in animals such as companion animals.

### BACKGROUND

According to the American Veterinary Dental Society (AVDS), 80% of dogs show oral disease by age 3, and it is the most common health problem treated in small animal health clinics today. The buildup of bacteria in a dog's mouth may cause more than just bad breath; according to research presented at a recent conference on Companion Animal Oral health, bacteria are also the cause of oral disease and diseases in other organs of the body like the heart, liver and kidneys.

As with humans, dog's teeth are prone to plaque buildup, and when allowed to combine with saliva and residual food between the tooth and gum, plaque turns to tartar. If plaque and tartar are not removed routinely by a veterinarian, they may cause periodontal disease such as gingivitis or periodontitis.

Salvadora persica is an evergreen drought-tolerant tree that grows very well on coastal sand dunes, marginal to high saline wastelands with or without water logging, ravines and saline/alkaline dry zones. During the initial 3 years of growth, S. persica requires some irrigation to establish the plant. Subsequently, minimal irrigation is needed. S. persica also can be irrigated with saline water. S. persica plants can yield 3.5 ton seeds/ha/year, and have a lifespan of up to 100 years.

### SUMMARY

The invention provides for S. persica compositions and articles of manufacture, as well as products containing an extract of S. persica. The compositions, articles of manufacture, and products are used for the oral health of companion animals.

In one aspect, the invention provides a flavored Salvadora persica stick. Representative flavorings include beef, pork, fish, bacon, and tuna.

In another aspect, the invention provides a S. persica stick that includes one or more medicines, one or more vitamins, one or more nutrients, or a flavoring. A representative medicine is an antibiotic.

In still another aspect, the invention provides an article of manufacture that includes at least one S. persica stick and a label indicating that the stick is for oral health of a companion animal. Representative companion animals include dogs, cats, and pot-bellied pigs. The S. persica sticks in an article of manufacture of the invention can include one or more medicines, one or more vitamins, one or more nutrients, and one or more flavorings.

In another aspect, the invention provides an article of manufacture that includes two or more S. persica sticks of uniform length and is labeled for use by companion animals. Typically, each stick is at least about 4 inches in length (e.g., about 6 inches in length, about 8 inches in length, about 10 inches in length, about 12 inches in length, or about 14 inches in length).

In another aspect, the invention provides an article of manufacture that includes at least five S. persica sticks that are each less than 4 inches in length and is labeled for use by companion animals.

In yet another aspect, the invention provides a product for chewing or ingesting by a companion animal that is effective for improving the oral health of a companion animal. Such a product includes an extract from S. persica sticks. Representative products include collagenous chewables, food, treats or snacks (e.g., a Greenie), and toys. Such products can be coated with the extract or impregnated with the extract.

In another aspect, the invention provides oral hygiene products for companion animals that include an extract from S. persica sticks. Oral hygiene products for companion animals include a toothbrush, toothpaste, and a medicament. By way of example, the bristles on a toothbrush can be coated or impregnated with the extract.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the drawings and detailed description, and from the claims.

### DESCRIPTION OF DRAWINGS

FIG. 1 is a photograph of a canine companion animal chewing on a S. persica stick.

### DETAILED DESCRIPTION

The invention provides a natural S. persica product for companion animals to chew on that improves the oral health of the animal. The benefits to companion animals of chewing on such a natural product have heretofore not been recognized. The products provided by the invention can beneficially reduce plaque, tartar, infections, periodontitis, and bad breath in companion animals. Therefore, the products of the invention can significantly reduce the extent of preventative dental treatment required by a companion animal, and also can eliminate the need for periodontal therapy and/or surgery.

S. persica and Extracts of S. persica

“Miswak” (or “siwak”) is an Arabic word that means tooth-cleaning stick. In English, miswak has been described as the “natural toothbrush.” The use of miswak by humans can be traced back at least to pre-Islamic times. Presently, many of the world populations including India, Pakistan, several African countries, the Arab countries, and most of the Muslim world still use miswak as an oral hygiene product. Miswak is a pencil-sized stick usually about 15-20 cm in length and 1-1.5 cm in diameter that is prepared from the root, stem, twigs or bark of any number of plants. The stick is chewed or tapered at one end until it becomes frayed into a brush. One of the primary sources for miswak is Salvadore persica (common name Arak or Araak), which grows in the Middle East and specifically around Mecca. Sticks from S. persica are widely used among Moslems after Prophet Mohammed (pbuh) realized its value as a device for cleaning teeth.

S. persica is a relatively small tree or shrub with a crooked trunk, seldom more than one foot in diameter. The bark is scabrous, cracked, and whitish with pendulous extremities. The root bark is light brown and the inner surfaces are white. S. persica has an odor similar to cress. S. persica can be grown in thorn shrubs, desert flood plains, and grassy savannahs. S. persica also can be grown in valleys, on dunes, and on termite mounds. S. persica can be found where ground water is readily available such as on river banks, on the perimeters of waterholes, in seasonally wet sites, and along drainage lines in arid zones. Table 1 shows the minimum and maximum altitude and rainfall limits for growing S. persica.

TABLE 1

| | |
|---|---|
| Minimum Altitude (m): | 0 |
| Maximum Altitude (m) | 1800 |
| Minimum Rainfall (mm): | 300 |
| Maximum Rainfall (mm): | 1000 |

The requirements for growing S. persica are as follows: S. persica is adapted to grow in alkaline or very saline soils that are usually clay-rich, and in soils lacking salt. S. persica prefers clays, but also can be found growing on loams, black soils, and sand. S. persica is extremely well-adapted to arid conditions, is salt tolerant, and is very resistant to drought conditions. S. persica readily germinates from seed (in about 24 hours) and also coppices well.

The fruit of S. persica are small, round, and pea-sized, and bear 1 seed per fruit. The seeds of S. persica turn from white to pink or purple-red, and are semi-transparent when mature. Seeds of S. persica exhibit no dormancy, but the fruit pulp contains germination inhibitors that should be removed before sowing. S. persica is usually grown in plantations or hedges, and is a slow growing plant. The S. persica tree has potential for reclaiming saline soils. Generally, the branches are cut repeatedly to produce short bushy plants with a number of stems.

Numerous beneficial ingredients have been identified in S. persica plants, including the following. S. persica contains antibacterial acidic inhibitors that fight decay and diarrhea. Antibacterial acidic inhibitors are natural disinfectants and can be used to stop bleeding. They disinfect the gums and teeth and close any microscopic cuts that may have existed in the gums. A mustard-like substance fights decay in the mouth and kills germs. S. persica also contains minerals such as sodium chloride, potassium, sodium bicarbonate, and calcium oxides, which clean the teeth. For instance, the American Dental Association (ADA) considers sodium bicarbonate to be a preferred ingredient in toothpastes. S. persica also contains natural scented oils that add taste and give the mouth a nice smell. In addition, S. persica contains enzymes that prevent the buildup of plaque that causes gum disease and loss of teeth. Further, S. persica contains anti-decay and anti-germ ingredients that decrease the number of bacteria in the mouth.

To identify and obtain any one or more of the above-described beneficial ingredients, the air-dried stem bark of S. persica can be chemically extracted with, for example, 80% alcohol, extracted with ether, and run through exhaustive chemical procedures. Previous experiments demonstrated that a particular S. persica extract is composed of trimethylamine, an alkaloid that may be salvadorine, chlorides, high amounts of fluoride and silica, sulphur, vitamin C, and small amounts of tannins, saponins, flavenoids, and sterols. Generally, an extract of S. persica can be obtained from the branches of the plant, although leaves, roots, and flowers also can be used.

There are many references in the literature that describe various extracts and extraction procedures. See, for example, Al lafi and Ababneh (1995, Int. Dent. J, 45:218-22); Darout et al. (2000, Ind. J. Pharm., 32:11-14); Ezmirly and El-Nasr (1981, J. Chem. Soc. Pak., 3:9-12); Gehlot and Bohra (2000, Curr. Sci. Bangalore, 78:780-1); Kamil and Ahmad (2000, Pak. J. Sci. Ind. Res., 43:255-7); Almas and Al (1999, Biomed. Lett., 60:71-5); Almas and Al (1997, Biomed. Lett., 56:145-9); Al and Al (1996, Biomed. Lett., 53:225-38); Al and Idowu (1994, Microbios., 80:107-13); Galletti and Chiavari (1993, Rap. Comm. Mass. Spec., 7:651-5); Sulaiman and Ajabnoor (1986, J. Ethnopharm., 17:263-8); and U.S. Pat. Nos. 6,428,823 and 6,586,021.

Extraction is a process whereby the desired constituents of a plant or plant part are removed using a solvent or other means. To produce an extract, plant material is usually first cleaned and dried if necessary. Drying can be done naturally (e.g., by air drying) or artificially (e.g., using warm-air fans or conveyor dryers). The plant material then can be ground, cut, or shredded using, for example, hammer action, pressure, friction or impact cutting. Methods of removing the desired constituents from the plant material include, but are not limited to, organic solvent extraction, supercritical gas extraction, and steam distillation.

There are a number of procedures for organic solvent extraction, including maceration (soaking and agitating the plant material with a solvent), percolation (repeated rinsing of the plant material with a solvent), and countercurrent extraction (continuous flow of a solvent in the opposite direction as the plant material). Representative solvents include, but are not limited to, ethanol, benzene, toluene and ether.

Aqueous extracts, such as decoctions (boiling the plant material, generally used for hard tissues), infusions (steeping the plant material, generally used for soft tissues) or macerations, can also be produced, although microbial contamination can be a concern with aqueous extraction methods. As used herein in the methods of the invention, a S. persica extract can be an ethanolic extract or an aqueous extract, depending upon the solubility of the active component(s) that improve oral health in companion animals.

Methods of producing active fractions (i.e., containing one or more active components) from a S. persica extract are provided by the invention. Active components of a S. persica extract or active fraction can include, but are not limited to, polyphenols, flavonoids, aromatic acids, metabolites, alkaloids, proteins, carbohydrates, starches, steroids, resins, elements or combinations thereof (e.g., glycoproteins) that, alone or in combination with other components, can improve oral health in companion animals. For example, fractionating by traditional solvent extraction employs partitioning of a solute between two immiscible phases, typically an organic phase (e.g., n-hexane, methylene dichloride, ethyl acetate or n-butanol) and an aqueous phase. Rapid extraction kinetics and the ability to utilize a number of different diluents, extractants, and aqueous phases makes solvent extraction a powerful separation method. In addition, numerous other separation procedures can be employed to further purify desired components or remove unwanted or contaminating components, including decanting, filtration, sedimentation, centrifugation, heating, adsorption, precipitation, chromatography, or ion exchange. The resulting active fraction can be subsequently concentrated by evaporation, vaporization, lyophilization or vacuum drying. Those of skill in the art are aware of the advantages of using certain separation techniques in combination with others to increasingly partition one or more active components into active fractions.

Compositions, Articles of Manufacture, and Products for Oral Health of Companion Animals

The invention provides for compositions, articles of manufacture, and products containing S. persica sticks or extracts for the oral health of companion animals. As used herein, companion animals refer to domesticated animals or animals that can be kept as pets. Companion animals include but are not limited to dogs, cats, pot-bellied pigs, guinea pigs, rabbits, horses, hamsters, gerbils, mice, rats, and ferrets.

Compositions of the invention can include S. persica sticks that are flavored or that contain one or more medicines, vitamins, and/or nutrients. Flavorings that can be used include, but are not limited to, beef, pork, fish, bacon, tuna, liver, or any other flavor that companion animals might favor. Medicines in general can include antibiotics, or anti-tick or -flea compounds. Alternatively, a medicine for use in a S. persica composition of the invention can be specific for a particular health issue (e.g., hypothyroidism) or can be a vaccine (e.g., a vaccine against Parvo or heartworm). Flavorings, medicines, vitamins, and/or nutrients can be applied topically to a S. persica stick, or the S. persica sticks can be soaked in a solution (e.g., aqueous or non-aqueous) containing the flavorings, medicines, vitamins, and/or nutrients.

The invention also provides for articles of manufacture containing at least one S. persica stick and a label indicating that the S. persica stick is to be used for oral health in companion animals. For an article of manufacture of the invention that contains two or more S. persica sticks, the sticks are typically of uniform (i.e., substantially the same) length. For example, the length of each S. persica stick in an article of manufacture of the invention can be about 4 inches, about 6 inches, about 8 inches, about 10 inches, about 12 inches, or about 14 inches. In one embodiment, an article of manufacture of the invention includes at least 5 S. persica sticks that are all less than 4 inches in length.

Articles of manufacture include packaging material. S. persica sticks can be packaged in bundles which are either tied or wrapped, packaged in boxes, or packaged in bags. Labels can be placed directly on the packaging material, or in some instances, can be printed directly onto one or more S. persica sticks using, for example, non-toxic, edible ink. Labels on articles of manufacture of the invention can indicate that the S. persica sticks are for oral health of companion animals. A label also can indicate if flavors, medicine, nutrients, and/or vitamins are present in/on the S. persica sticks. If, for example, medicines are present in/on the S. persica sticks, the label may indicate how many sticks a companion animal should be allowed to chew or ingest in a given period of time (e.g., 24 hours).

The invention additionally provides products containing a S. persica extract. Such products are effective for improving the oral health of a companion animal. Products for companion animals that can contain an extract of S. persica include, but are not limited to: collagenous chewables, food, treats or snacks, and toys. Collagenous chewables include, for example, pigs feet and pigs ears. Food for a companion animal can be dry food or a moist, soft food. Treats or snacks for companion animals include rawhides, Greenies®, Milkbones®, Friskies®, Whiskas®, and Pounce® treats, for example. Toys for companion animals can include balls, robes, or toys to chew or gnaw on. A S. persica extract can be applied topically (e.g., coated on) to the above-mentioned products, or can be impregnated within the product (e.g., by soaking the product in the extract, or by introducing the extract into the product during the manufacturing process).

A product of the invention that contains an extract of S. persica also includes those products used in the oral hygiene of a companion animal. For example, S. persica extracts can be incorporated into the bristles of a companion animal's toothbrush (see, for example, U.S. Pat. No. 6,490,747), into toothpaste for use by a companion animal (see, for example, U.S. Pat. Nos. 5,009,886 and 6,264,926), or into an oral medicament for use by a companion animal. According to the invention, the bristles of a toothbrush can be coated with the extract before or after the bristles are attached to the handle of the toothbrush or, alternatively, the bristles can be impregnated with the extract during the manufacturing process.

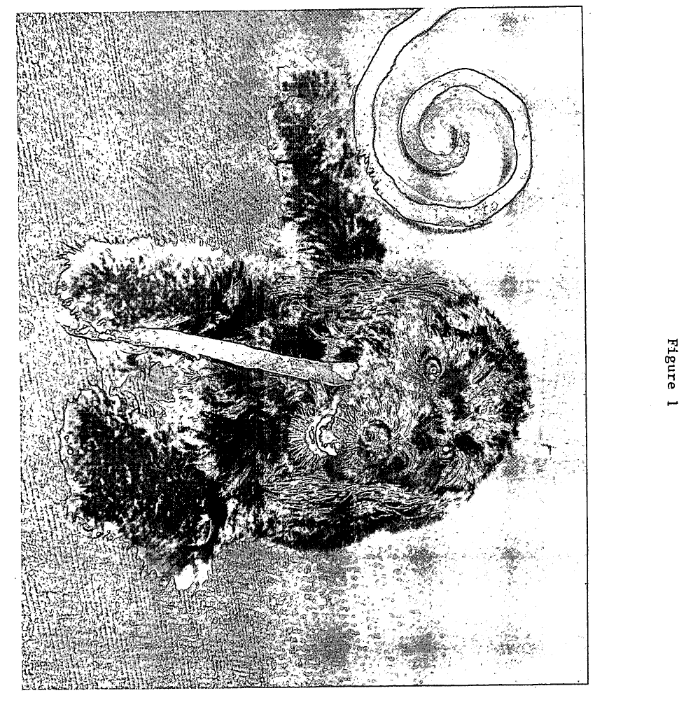

As shown in FIG. 1, S. persica sticks are enjoyed by dogs. S. persica sticks can provide numerous benefits in oral health of companion animals, but can also be used to deliver medicine, vitamins or nutrients. S. persica sticks additionally provide very little, if any, calories.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A flavored Salvadora persica stick.

2. The flavored S. persica stick of claim 1, wherein said flavoring is selected from the group consisting of beef, pork, fish, bacon, liver, and tuna flavorings.

3. A S. persica stick comprising one or more medicines, one or more vitamins, one or more nutrients, or a flavoring.

4. The S. persica stick of claim 3, wherein said medicine is an antibiotic.

5. An article of manufacture comprising at least one S. persica stick and a label, wherein said label indicates that said stick is for oral health of a companion animal.

6. The article of manufacture of claim 5, wherein said companion animal is selected from the group consisting of a dog, a cat, a pot-bellied pig, a guinea pig, a rabbit, a horse, a hamster, a gerbil, a mouse, a rat, and a ferret.

7. The article of manufacture of claim 5, wherein said stick comprises one or more medicines, one or more vitamins, one or more nutrients, and one or more flavorings.

8. An article of manufacture comprising two or more S. persica sticks of uniform length, wherein each stick is at least about 4 inches in length, wherein said composition is labeled for use by companion animals.

9. The article of manufacture of claim 8, wherein each stick is about 4 inches in length.

10. The article of manufacture of claim 8, wherein each stick is about 6 inches in length.

11. The article of manufacture of claim 8, wherein each stick is about 8 inches in length.

12. The article of manufacture of claim 8, wherein each stick is about 10 inches in length.

13. The article of manufacture of claim 8, wherein each stick is about 12 inches in length.

14. The article of manufacture of claim 8, wherein each stick is about 14 inches in length.

15. An article of manufacture comprising at least five S. persica sticks, wherein each stick is less than 4 inches in length, wherein said article of manufacture is labeled for use by companion animals.

16. A product for chewing or ingesting by a companion animal, said product comprising an extract from S. persica sticks, wherein said extract is effective for improving the oral health of a companion animal.

17. The product of claim 16, wherein said product is selected from the group consisting of collagenous chewables, food, treats or snacks, and toys.

18. The product of claim 16, wherein said treat is a Greenie.

19. The product of claim 16, wherein said product is coated with said extract.

20. The product of claim 16, wherein said product is impregnated with said extract.

21. An oral hygiene product for companion animals, wherein said oral hygiene product comprises an extract from S. persica sticks.

22. The oral hygiene product of claim 21, wherein said oral hygiene product is a toothbrush.

23. The oral hygiene product of claim 22, wherein bristles of said toothbrush are coated with said extract.

24. The oral hygiene product of claim 22, wherein bristles of said toothbrush are impregnated with said extract.

25. The oral hygiene product of claim 21, wherein said oral hygiene product is toothpaste.

26. The oral hygiene product of claim 21, wherein said oral hygiene product is a medicament.
